(19) United States
(12) Patent Application Publication
(10) Pub. No.: US 2011/0166572 A1
(43) Pub. Date: Jul. 7, 2011

(54) LATERAL IMPLANT SYSTEM AND APPARATUS FOR REDUCTION AND RECONSTRUCTION

(75) Inventor: Stefan Ihde, Uetilburg (CH)

(73) Assignee: BIOMED EST, Vaduz (LI)

(21) Appl. No.: 13/017,370

(22) Filed: Jan. 31, 2011

Related U.S. Application Data
(62) Division of application No. 11/506,614, filed on Aug. 18, 2006.
(60) Provisional application No. 60/709,232, filed on Aug. 18, 2005.
(60) Provisional application No. 60/709,233, filed on Aug. 18, 2005.
(60) Provisional application No. 60/740,098, filed on Nov. 28, 2005.
(60) Provisional application No. 60/757,194, filed on Jan. 6, 2006.

(30) Foreign Application Priority Data
Mar. 7, 2006 (DE) 202006003922.8
Apr. 25, 2006 (DE) 202006006920.8
May 24, 2006 (DE) 202006008702.8
Jun. 27, 2006 (DE) 202006010202.7

(51) Int. Cl. A61B 17/80

(52) U.S. Cl. 606/70; 606/286

(57) ABSTRACT

A bone fixation apparatus and method includes basal implants dimensioned to be installed in bone through lateral insertion into a T-shaped slot. The implants serve as anchors for mounting plates to be placed on either side of a fracture. The mounting plates or anchors may be a mount to which a stabilizing fixation rod, plate, prosthesis, dental prosthesis or other mesiostructure is attached.

### CROSS-REFERENCE TO RELATED APPLICATIONS

This application claims priority to U.S. Provisional Patent Applications 60/709,232 filed Aug. 18, 2005; 60/709,233 filed Aug. 18, 2005; 60/740,098 filed Nov. 28, 2005 and 60/757,194 filed Jan. 6, 2006; and to German Patent Application No. 20 2006 006 920.8 filed Apr. 25, 2006; German Patent Application No. 20 2006 010 202.7 filed Jun. 27, 2006; German Patent Application No. 20 2006 008 702.8 filed May 24, 2006; and German Patent Application No. 20 2006 003 922.8 filed Mar. 7, 2006.

### BACKGROUND OF THE INVENTION

1. Field of the Invention

The present invention is in the medical field of reduction and fixation of long bone fractures, human mandible fractures and anchoring prosthetics and maxillofacial implants, in particular implants following surgical resections.

2. Related Art

Fixation and splinting of fractured bones has long been a challenge for medical and dental practitioners. There has been a constant need for secure fixation to allow for mending of bones while simultaneously providing for the earliest possible return to function of the broken bone.

These problems are particularly true in oral or maxilo-facial area, where chewing can exert strong and often eccentric forces on the mandible and/or maxilla. These forces have long made it a challenge to achieve early return to function for a patient suffering from a broken mandible or maxilla.

Lateral implants for dental uses have been previously disclosed in U.S. Pat. No. 6,402,516 and U.S. patent application Nos. 60/740,098, 10/163,034, 60/709,233 and 11/105,944 all of which are incorporated by reference herein.

In particular, familiar problems are even further exacerbated in the case of the edentulous mandible. The prior approach is to fixation of a broken mandible generally include using two plates or using a single large plate which plate provides for a relatively large number of holes, for example 10 to 15 in order to accommodate the many screws or pins needed to fix the broken bone.

Separately, in the prior art unbroken edentulous mandibles were most frequently treated with a full denture. Chewing with a full denture is suboptimal and frequently problematic, since fixation of the denture to the lower mandible and gums is seldom fully sufficient and typically allows the denture to “float” in the oral cavity during chewing. Accordingly, these patients have a separate need which may be more adequately addressed with a full fixed bridge.

Further, one common prior approach to installing bridges in the edentulous mandible was to use a screw-type implant. Screw-type implants are problematic in that in order to function properly, they require a certain depth of bone to be available. This mandible height is frequently unavailable in the edentulous mandible.

For the patient who fractures an endentulous mandible, there does not currently exist a fully optimized, durable, economic, relatively easy to install solution that promotes rapid mending of the fractured mandible while further promoting a rapid return to full function.

With surgery in general and in particular the field of maxillofacial surgery a recurring circumstance is the need to resection bone, tissue and organs surgically in order to remove tumors. On occasion, trauma can also generate the need for related surgery. Although such surgeries may be life saving, the resulting large facial defects have a serious cosmetic impact for the patient. There is a continuing need in the art for more durable, more efficient and more readily and quickly deployable anchoring systems for prosthetic devices to ameliorate these cosmetic effects.

Radiation therapy commonly follows tumor resections, especially those that are performed in the orbita and/or the nose. Radiation therapy affects the ability of the bone to carry implants. Experience has shown that during or after radiation therapy conventional screw implants have suffered very low success rates, due to implant rejections caused by osteonecrosis, osteomyelitis and the like. Previously, only prolonged waiting periods of up to 24 months will lower the failure rate experienced with conventional screw implants. For the patient with a substantial cosmetic defect, this waiting period is difficult. The high failure rate is caused by BMU osteosystems which remodel internal bone structure under normal circumstances being destroyed by the radiation and do not regenerate quickly. There is a need in the art for an implant that can be used to support prosthetic devices for these patients more quickly after radiation therapy is finished, or even during radiation therapy.

Dental implants have been inserted entirely into the alveolar crest. There is a need in the art for a dental implant that does not have to be inserted entirely into the alveolar crest.

Buser et al already showed a tent function, but not in combination with a (lateral) implant being the “holding way” device: Europäischen Patentschrift 0 504 103 B1.

In orthopedics when setting fractures of long bones, the stability of fixation hardware is of critical importance. The prior art has traditionally used screws for anchoring plates and other fixation hardware to stable portions of a reduced long bone fracture for anchoring other fixation equipment. While moderately successful, there is a constant need in the art for maximizing the stability of fixation hardware anchors.

Hardware anchoring also needs to be braced against stresses in numerous angles to the greatest degree possible. In order to achieve multi-angle stress stability, many screws are needed using the prior art. This has the disadvantage of degrading the structural integrity of the bone in which the anchors are placed. Maximizing the range of angular stability while minimizing the number of anchors used is a present need in the art.

Other problems with prior art devices include the pin coming out of the bone on areas that are not so strong (low mineralization areas) and where prior art screws would not adequately hold. Bone does not form around the endosseous parts of screw implants or fixation screws, especially in osteoporotic bone. There is a need to promote woven bone in addition to the existing cortical bone, so there is more bone in the end. Infection is always a complication to be resisted. There is a need in the art for a device that is stronger, fights infection better and promoted greater bone growth, in particular woven bone growth. There is also a need to keep the cut made in the bone to receive the lateral implant as narrow as possible so that healing and re-closure of that implant bed be achieved as rapidly as possible. However, the vertical shaft or post of the implant must remain thick enough so that it does not break in use.

Coating

The invention also concerns a coating used in orthopedic surgery, and in dental and maxillofacial implantology, especially for enossal implants.

Maintaining the stability of enossal implants with respect to the bone into which they are placed is often a clinical problem. Mobility of implants is often observed both in orthopedic surgery and in dental and maxillofacial implantology. A certain portion of that mobility is due to infection. However, most of the mobility is caused by overloading the peri-implant bone. For instance, it is the most highly stressed screws, or the screws positioned in the least mineralized regions, such as in the tension or flexion regions of the bone, that become mobile in the case of fracture osteotomy plates.

The measures that have been known to limit or prevent these undesired processes amount to promoting new bone formation in the bony surgical region. Thus it has been suggested, among other things, to accelerate and stimulate the formation of new bony tissue by coating the implant surface with substances that promote bone growth.

Such procedures, and recommendations for coating of implants, are, for instance, known from DE 600 19 752 T2, DE 196 30 034 A1 and DE 196 28 464 A1. The measures known so far for coating implants relate predominantly to improved preparation of substrates for bone development, such as tricalcium phosphate, hydroxylapatite, and all sorts of calcium and phosphorus compounds. Measures for improved blood supply to the bone were also recommended to accelerate and stimulate formation of new bone tissue. Finally, increased provision of growth hormones and peptides of all types, which accelerate bone development, have been recommended.

None of those efforts has yet resulted in an actual measurable clinical result, and there has been no overwhelming success in clinical practice, as it takes many weeks to months before the newly formed bone truly mineralizes and becomes capable of bearing a load. The implant mobility mentioned occurs much sooner, though.

Therefore, the invention is based on the objective of creating a microtherapeutic reduction of the osteonal activity in the immediate vicinity of enossal implants by an altered coating, thus preventing destabilization of enossal implants.

### SUMMARY OF THE INVENTION

A bone fixation apparatus and method includes basal implants dimensioned to be installed in bone through lateral insertion into a T-shaped slot. The implants may serve as anchors for mounting plates to be placed on either side of a fracture. A stabilizing fixation rod or other device may be attached to the mounting plates.

The present invention includes a system, apparatus and method that may be advantageously used for fixation of oral maxillo-facial fractures, particularly in the case of the edentulous mandible fracture. The system comprises a full fixed bridge, at least two lateral implant devices and a plate with screws or pins. The lateral implants to be used are characterized by non-screw type seating in particular anti-rotational seating. Appropriate lateral implants are more fully described in U.S. patent application Ser. Nos. 10/163,034; 11/105,944; 11/015,548 and 10/714,200, which are incorporated by reference as fully set forth herein. The method of use of the invention is to install a reduced size plate straddling the fracture and use standard pins or screws to anchor the plate and thereby fix the mandible with its fracture components reduced to their proximated positions. Thereafter at least one T-shaped slot is installed on a first side of the mandible fracture to receive a lateral implant and at least one second such slot is created on a second side of the fractured mandible. A full fixed bridge is installed and securely anchored to the two or more lateral implants. In this manner, a more rapid return to function is possible while simultaneously providing a secure fixation of the fractured mandible for healing. After full healing is achieved, the plate and its screws or pins may be removed. The bridge and its implant mounts are left in place. Thereby, the patient has the double advantage of his fracture having been treated and also the continuing presence in his mouth of the bridge.

The implant does not have to be inserted into the alveolar crest completely, but only with the base plates that show into the bone direction. Then augmentation material, resorbable or not resorbable, can be augmented and the shape of the augmented site is given by the baseplates; then a fibrin-membrane (made from the patients blood) or any other membrane (artificial, cow, pig, other origins, etc), can be put over the exposed baseplates and enhance healing.

This system, apparatus and method may also be used for anchoring prosthetic devices. The apparatus and system of the present invention uses lateral implants. During insertion of the lateral implants, large T-shaped slots are created within the bone, and may include the radiated bone. These slots fill with blood, which from the natural process of stem cell development turns into callus woven bone. These cells initiating new bone formation are not affected by the local radiation therapy. The system and method of the present invention may include fibrin membranes being placed around the implants.

Lateral implants distribute the forces to bone areas which are strong (highly mineralized), as opposed to prior art pins that come out of the bone on areas that are not so strong (low mineralization areas) and where screws would not adequately hold. Of course, the present system may be used in combination with conventional screws for fixation. The open slots of the present system promote woven bone formation, especially in osteoporotic bone. Woven bone is created in addition to the existing cortical bone, so there is a more bone in the end. The pins used herein may be completely smooth so infection can not catch easily as in screw implants.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 depicts a fractured bone and basal implants.

FIG. 2 depicts a reduced bone fracture with basal implants and mesiostructures.

FIG. 3 depicts a reduced tibial fracture with mesiostructures in place and a long bone plate.

FIG. 4 depicts a reduced tibial fracture with all structures in place.

FIG. 5 is an alternative embodiment of the screw/mesiostructure connection.

FIG. 6 depicts a basal implant.

In FIG. 7 depicts a basal implant with a female threading.

FIG. 8 depicts a basal implant with an abutment terminus.

FIG. 9 depicts an alternate female threading arrangement of a basal implant post.

FIG. 10 depicts abutments.

FIG. 11 depicts adjustable plate placement FIG. 12 depicts alternative basal implants.

FIG. 13 is a perspective view of a fractured mandible.

FIG. 14A is a perspective view of the fractured mandible, with the hardware of the present system and method shown in exploded view.

FIG. 14B is a perspective view of the fractured mandible reduced and with implant slots cut.

FIG. 14C is a perspective view of the mandible with the fracture plated before implant slots are cut.

FIG. 14D is a perspective view of a fractured mandible shown with alternative placements of an implant.

FIG. 14E is a close up of alternative implant placement.

FIG. 15 is a perspective view of the mandible with the implants of the present system in place.

FIG. 16 is a perspective view of the mandible with all of the hardware of the present invention in place.

FIG. 17 is a perspective view of a basal implant.

FIG. 18 is a perspective view of an alternative embodiment of a basal implant.

FIG. 19 is a perspective view of the fractured mandible reduced and with implant slots cut.

FIG. 20A shows a first step in alveolar augmentation.

FIG. 20B shows another alveolar augmentation alternative.

FIG. 20C shows another alveolar augmentation alternative.

FIG. 20D shows another alveolar augmentation alternative.

FIGS. 21A, 21B and 21C show basal implants.

FIG. 22 is a perspective view of a model skull showing the installed apparatus over an eye socket and a partially installed apparatus over the sinus.

FIG. 23 is a close up perspective view of a model skull showing the installed apparatus over an eye socket and a partially installed apparatus over the sinus.

FIG. 24 is a front view of the assembly of the present invention installed in the sinus.

FIG. 25 is a front view of a partially completed installation of the present invention over the sinus.

FIG. 26 depicts various lateral implants.

FIG. 27 is an exploded view of an implant, implant slot and fibrin membrane.

FIG. 28 is a schematic representation of the implant according to the invention.

FIG. 29 is the section A-A as indicated in FIG. 28.

FIG. 30 is a view of a partial base plate.

FIG. 31 is a view of the partial base plate.

FIG. 32 is a view of the partial base plate.

FIG. 33 is a view of the partial base plate.

FIG. 34 is a view of the partial base plate.

FIG. 35 is a view of the partial base plate.

FIG. 36 is a view of the partial base plate.

FIG. 37 is a view of the partial base plate.

FIG. 38 is a view of the partial base plate.

### DETAILED DESCRIPTION OF THE PREFERRED EMBODIMENTS

The following description of the preferred embodiment(s) is merely exemplary in nature and is in no way intended to limit the invention, its application, or uses.

Long Bone Fixation

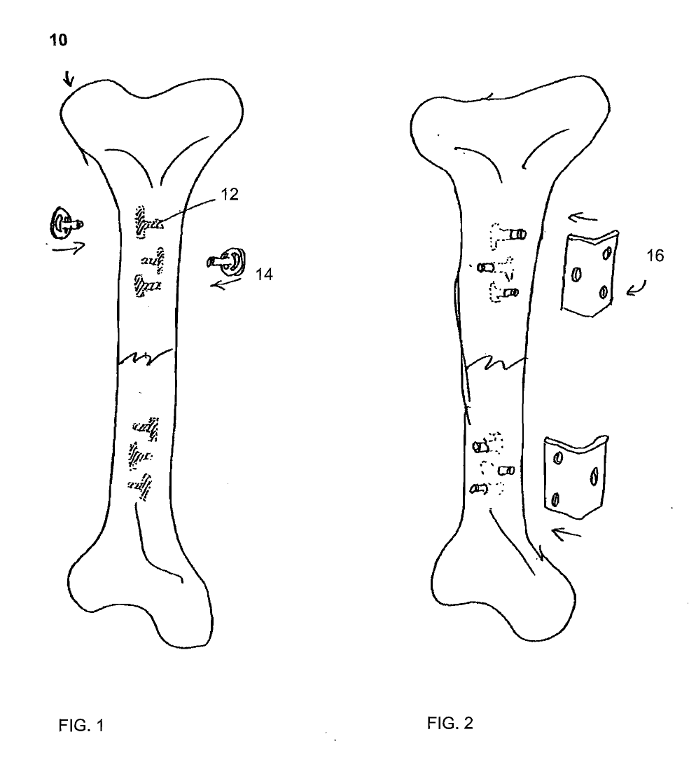

Referring to the figures where like reference numbers indicate like elements, a long bone such as tibia 10 presenting with a fracture 8 depicted in FIG. 1. Also depicted are basal implants 14, described in greater detail below. They have the common characteristic of having a base plate and a perpendicular rod. In installation, the orthopedic surgeon would cut T-shaped slots 12 in the bone. Advantageously, multiple slots at each anchor site may be cut and oriented such that the basal implants will insert and mount at various angles.

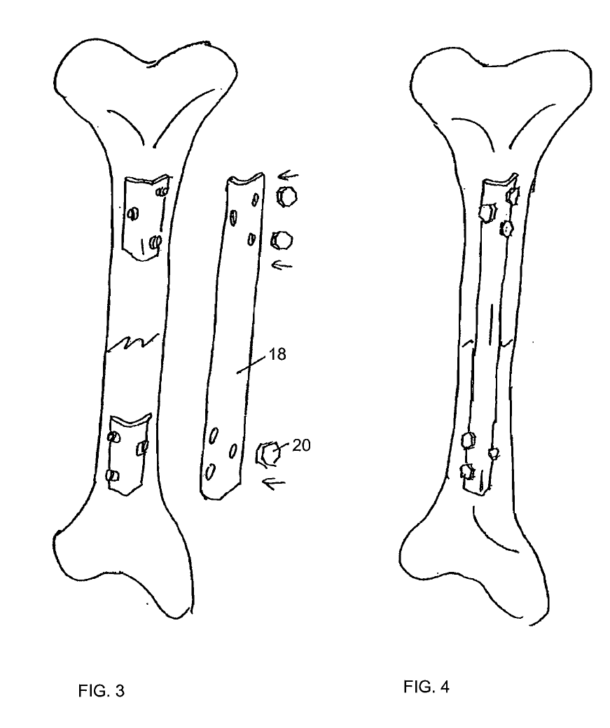

As depicted in FIG. 2, the basal implants are inserted into the slots 12 and a top of a post of the basal implant extends beyond the surface of the bone and outwards to receive further hardware. The implants may optionally be secured with screws as well. This hardware will include intermediate fixation plates referred to herein as mesiostructures 16. These mesiostructures may be of various materials, for example metal, particularly stainless surgical steel. Mesiostructures may further be fabricated in a wide variety of shapes and sizes. Optimally, a variety of standard shapes will approximate a curvature of known human bone sites that commonly present as anchors sites for long bone fractures. The mesiostructures are selected in tandem with the type of basal implants 14 to be used. Accordingly, a series of throughholes 17 in the mesiostructure 16 or other anchoring structures described more fully below are preconfigured to match with the basal implant 14 posts extending from their insertion sites. It is within the scope of the present invention that mesiostructures 16 may be custom designed, or even malleable enough for manipulation during installation. They may be prefabricated and custom fit, by hand or with a computer model of the patient's bone. The mesiostructures and/or the fixation plates could be fabricated of self-setting or light curing materials such as acrylates, composites, etc. Alternatively, the throughholes 17 may be oblong or otherwise accommodate adjustment to minor variations in the angle of the basal implant posts to which they will be affixed.

FIG. 3 depicts the mesiostructures installed over the extending basal implant posts. Thereafter, a long plate 18 will be installed over the mesiostructures 16. Long plate 18 also has throughholes 19 predisposed to mate with the extending basal implant posts. Finally, cap nuts 20 may be placed over the terminal ends of the extending basal implant posts and fixed thereto, as for example by screwing onto them. FIG. 4 depicts the assembly of the present invention fully installed.

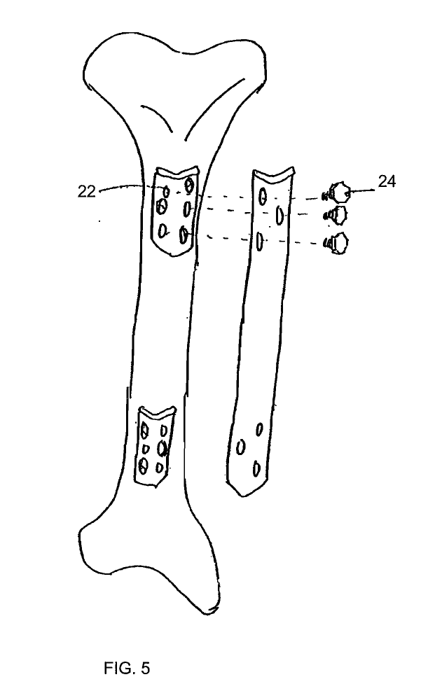

FIG. 5 depicts an alternate embodiment. Here, the extending basal implant posts are preconfigured to extend only as far the outer surface of the mesiostructures. They are screwed in place there with for example, male threaded bolts preconfigured to mate with female threaded posts of the basal implants. Interspersed between the mesiostructure anchoring throughholes are outer plate anchoring countersinks 22. They are designed to receive a male screw 24. In final assembly, the long plate 18 is placed over the mesiostructures in place, as before, but the long plate is anchored to the mesiostructures with a second set of screws, or bolts 24 by screwing into the countersinks 22 provided for them on the outer surface of the mesiostructures. The term mesiostructures as used herein may also include dental prosthetics, stabilizers, and reconstructive appliances.

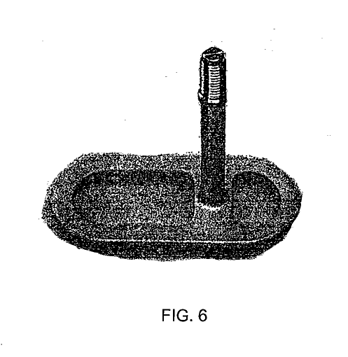

FIG. 6 depicts a basal implant. It is characterized by a base plate 30 on a first plane and a post 32 perpendicular to that plane. The base plate is often oblong, which aids in stability and in arresting rotation. The base plate often has prefabricated structural through holes or spaces 34, to aid in osseointegration.

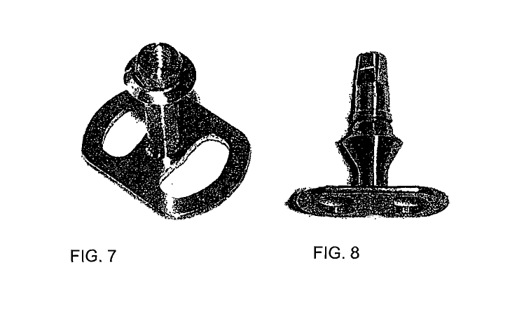

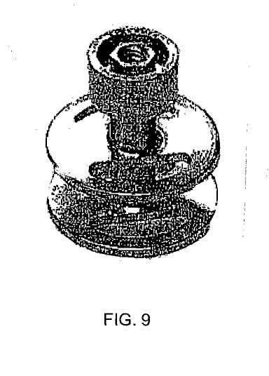

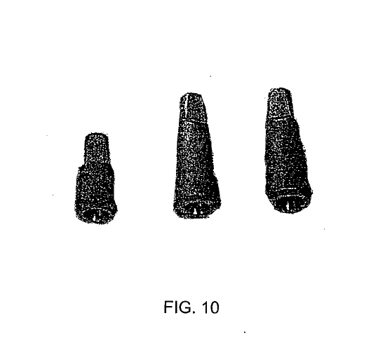

The basal implant depicted in FIG. 6 has a male threaded post. In FIG. 7 a basal implant with a female threading is depicted. In FIG. 8 a basal implant with an abutment terminus is depicted. FIG. 9 depicts an alternate female threading arrangement of a basal implant post. FIG. 9 is also notable for having two anchoring plates, thereby further augmenting stability. FIG. 10 depicts abutments which are configured to mate with abutments such as that shown in FIG. 8.

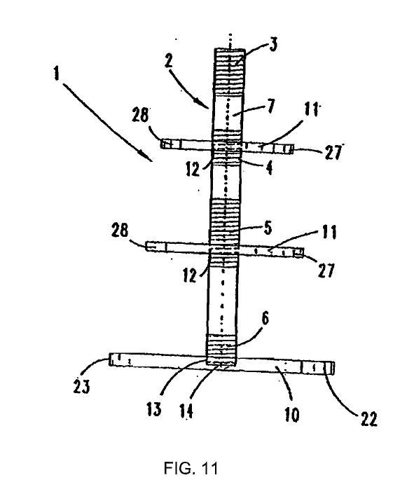

FIG. 11 depicts adjustable plate placement, thereby affording the orthopedic surgeon further flexibility in adapting a basal implant to the shape of the bone available for an anchor site. FIG. 12 further shows a basal implant with a further stability extension 36. FIG. 12 depicts a flexible basal implant.

The different parts of the implants can be manufactured from different materials which are soldered or screwed together. The advantage of this is that the intrabony part can be made from titanium which is highly biocompatible for bone, but not so easy to clean outside of the bone. Stainless steel or other easy to polish and clean material can penetrate/project through the skin or mucosa because in lateral implants the vertical implant parts are not necessarily osseo-integrated.

Mandible Fixation:

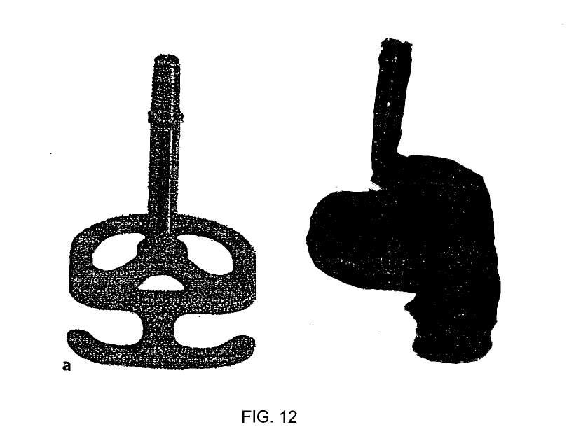

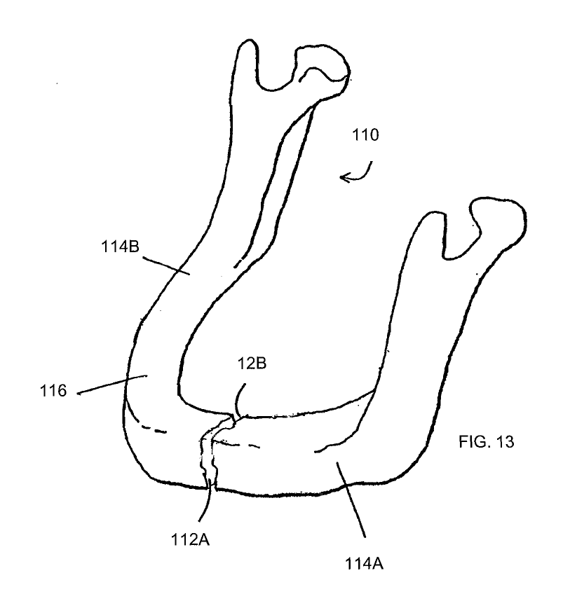

FIG. 13 is a perspective view of a fractured human mandible. Depicted is an edentulous mandible. Edentulous mandibles represent particular problems for these implants and screw modification devices. Moreover, patients suffering from osteoporosis have reduced bone mass and present similar problems. These items include the lack or reduced volume of bone sufficient for flexing and fixation and maintenance of implants.

Present in FIG. 13 is the mandible 110, fracture faces 112a and 112b and a pair of molar implant sites 114a, 114b and a canine implant site 116.

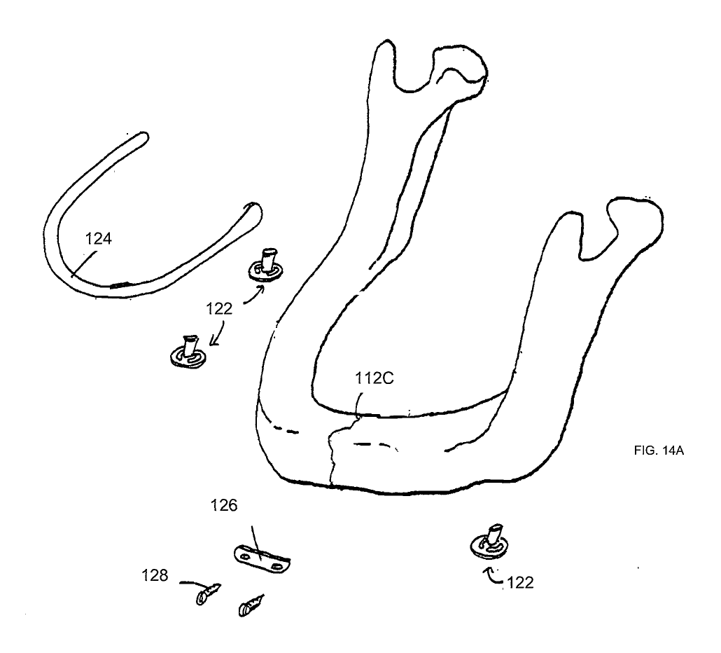

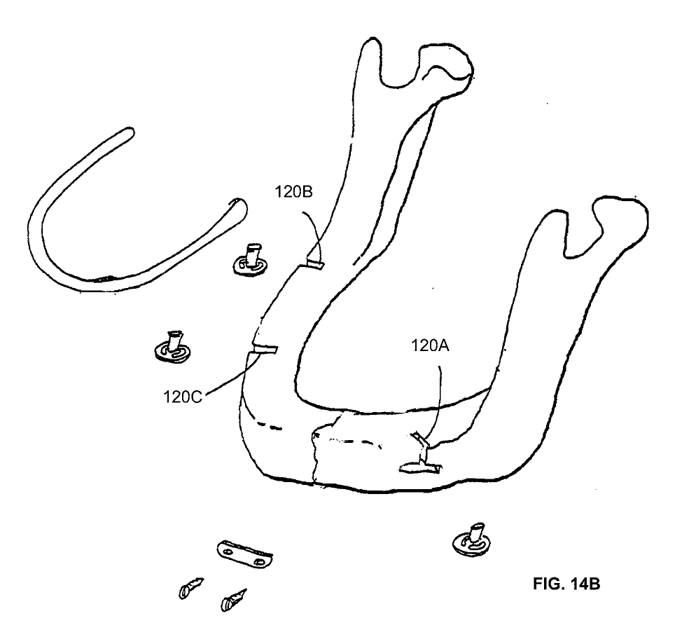

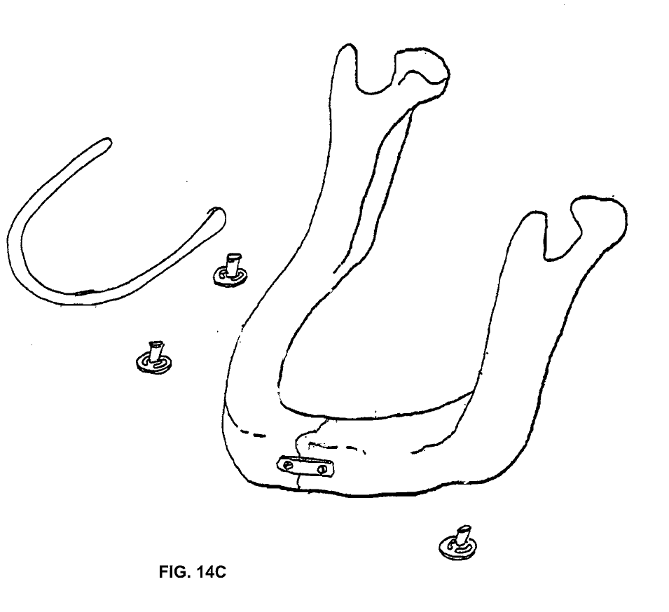

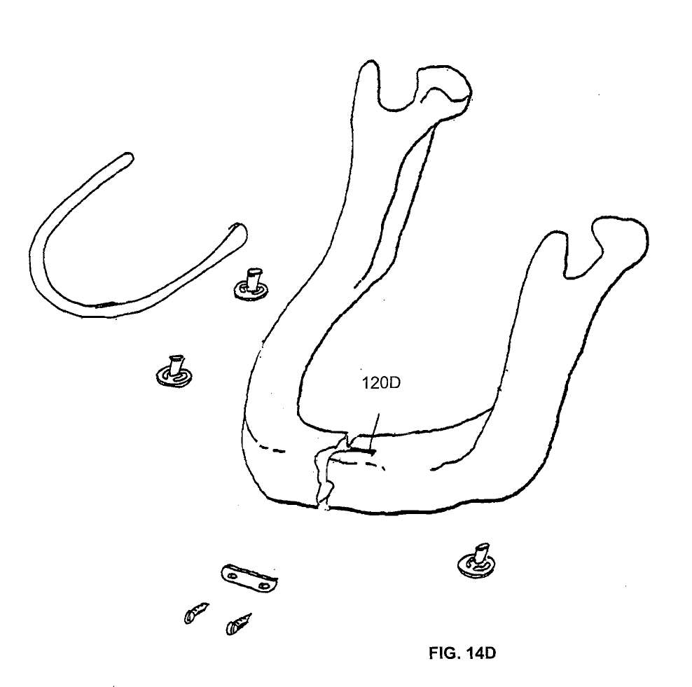

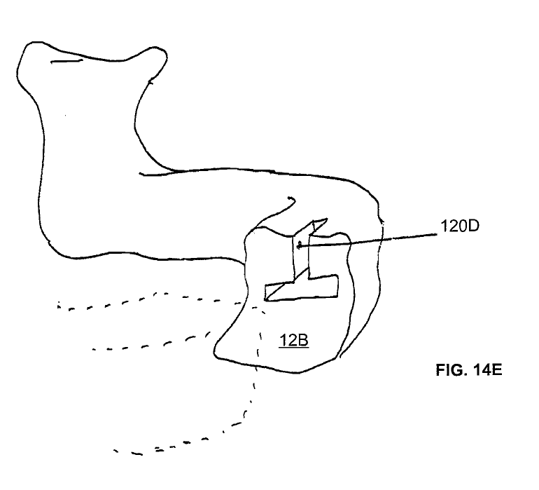

In FIG. 14, the hardware of the present system is depicted. This includes implants 122 which may be used for anchoring crowns, individual teeth, bridges or full dentures, as well as the bar 124 shown in FIG. 14. Finally, a reduced surface area plate 126 together with screws for mounting it 128 is depicted.

Depicted in FIG. 14A are slots 120a, 120b and 120C which are cut in particular locations in the mandible by the dentist or maxillofacial surgeon for insertion of the implants. Slots 120a, 120b are molar slots corresponding to the area in which the patient's molars have been historically. Slot 120c is a canine location. These slot locations are strategic positions because they optimize the balance of strong fixation of the fracture site together with the earliest return to full function. These positions are preferred for implants also because of the biomechanics of occlusion. Finally, these sites correspond to the sides of greatest boney mass density in osteoporitic patients and also avoid other sensitive anatomy such as vascular and nerve pathways.

The implants used are non-screw type, T-shaped or double T-shaped implants. Further, it is advantageous to use non-rotational type implants. Such implants are further described in U.S. patent application Ser. Nos. 09/829,351; 10/163,034 and 11/105,944 which are incorporated by reference as fully set forth herein.

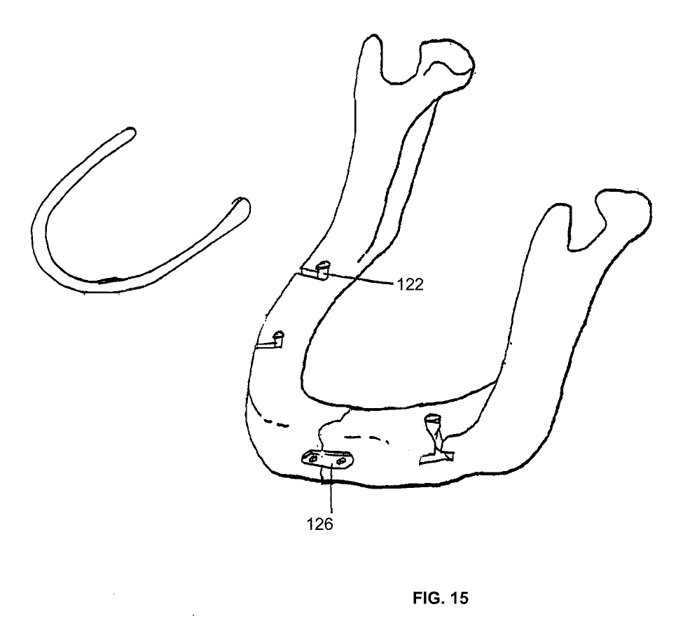

The clinician's approach is to reduce the fracture by approximating the fracture faces 112a, 112b. Thereafter, in the embodiment depicted in FIG. 14C, slots are cut into the mandible for receiving the implants. In a fracture offset from the midline as depicted here, at least one implant would be placed on a “short” side of the mandible, preferably in one of the strategic positions, which in FIGS. 14A and 15 is a molar position. Alternatively, as depicted in FIG. 14C, the fracture 112c may be plated before slots are cut.

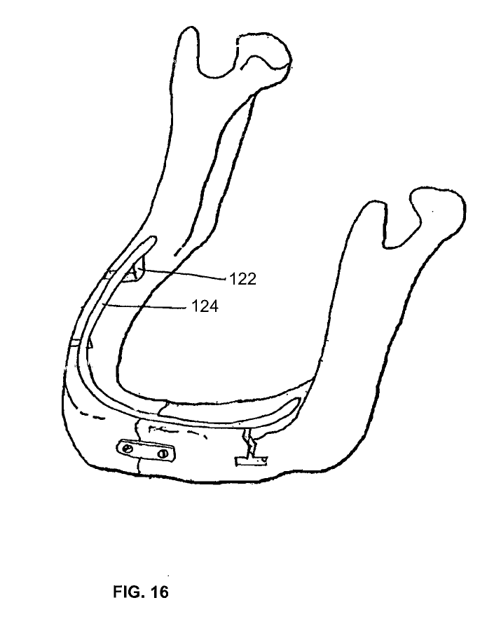

In the depicted embodiment, at least two implants would be placed on the opposite or “long” side of the mandible. In the depicted embodiment, two implants are shown at a molar 114A, 114B and at a canine position 114C. The clinician also places a plate 126 dimensioned to straddle the actual fracture line 112c and to receive screws or pins 128, at least one on either side of the fracture line 112c, in order to fix it. Having reduced the fracture, plated it, and cut the slots for receiving implants, a practitioner next places the implants and rotates them into place as shown in FIG. 15. Finally, the bar 124 for supporting a dental plate is installed onto the implant upright as shown in FIG. 16.

FIGS. 14D and 14E depict an alternative embodiment of the present invention. Therein, the actual face of the fracture 112a or 112b is used as the site for cutting a slot 120D for implant seating. In this manner fixation is had directly at the fracture site. Further, the fracture site will help promote bleeding and blood flow around the actual implant. Blood flow is advantageous for fixation of implants in that a blood filled space within a bone transforms into organized or woven fibers in a short period of time. These fibers organize and around the implant itself and in time calcify. In this way, the implant is more securely fixed into position. Woven bone exhibits good mechanical properties and advantageously secures the interlocking of the fractured bone segments to stabilize the fracture itself. Such calcification of bone forming from a blood clot is known to be more highly mineralized than the original bone and promotes a stronger splint at the fracture site.

Accordingly, the practitioner proceeding along the lines depicted in FIG. 14D will first cut the slot on the fracture face 112c, and install an implant in that particular slot. Thereafter, the practitioner will either cut the other slots for the implants, install a plate and then reduce the fracture completely, or alternatively reduce the fracture completely, and then cut the slots of other implants and install them.

Bar 124 serves as a mount for artificial teeth. These may be mounted during or after healing. After the mandible has healed, the plate and pins are removed. The implants remain in place and the bar maintains the position of the dentures attached to it.

The system and method of the present invention is flexible. For a midline fracture, two basal implants in the area of the canines and two in the area of the second molars are used. In the case of the non-midline fracture, one basal implant may be positioned on the “short” side of the fracture and two or more placed on the other side of the fracture. Alternatively, two basal implants may be placed on the short side of the fracture and three or four on the opposite sides.

It is a further aspect of the present invention that the bridge placed on the implant mounting shafts may be changed. In this way, an initial bridge may be used to promote healing of the fracture by having a first occlusion profile and a final bridge may be used after healing having a final occlusion profile. In the preferred application of the invention, base plates are used distal of the mandibular nerve, taking into account the usually reduced volume of bone, particularly in the vertical dimension that is available for fixation or implant hardware.

The invention may be further applied in combination with the addition of crestal implants (screw implants) especially into the anterior part of the jaw bone, either upper or lower to support the lateral implants. Finally, the healing process may be supplemented by the use of applying botulinum toxin into the masticatory muscles (the masseter and temporalis) in order to reduce the forces generated by chewing on the fracture site. See, U.S. Application Ser. No. 60/671,024, which is incorporated by reference as if fully set forth herein.

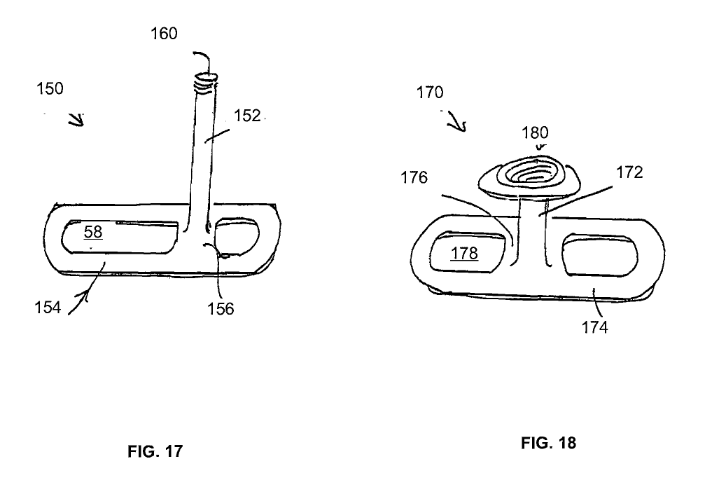

FIGS. 17 and 18 depict exemplary alternative embodiments of basal implants. Basal implants 150 and 170 are characterized by posts 152 and 172 with a fixation device on top of them. The bottom of the post is anchored to lateral portions 154 and 174. The lateral portion is substantially perpendicular to the post in the depicted embodiments. The lateral portion may be symmetrical on either side of the post, as in the embodiment depicted in FIG. 18. Alternatively, the lateral portion may be asymmetrical on either side of the post, as depicted in FIG. 17. The lateral portion has a center cross member 156 and 176 for connection to the posts 152 and 172. The lateral portions are further characterized by spaces 158 and 178 defining an outer boundary of the lateral portions 154 and 174. These outer boundaries in the depicted embodiments are further characterized by extending more widely than the diameter of the posts 152 and 172 in all directions substantially perpendicular to posts 152 and 172. Stated alternatively, the basal implants are dimensioned for lateral installation through substantially T-shaped slots made in the bone by the practitioner.

The basal implant embodiments depicted in FIGS. 17 and 18 are different in that post 152 has an externally threaded fixation appliance 160 at its top. The embodiment depicted in FIG. 18 has an internally threaded, concave, female fixation appliance 180 at its top. In this manner a wide variety of attachment devices for fixedly connecting the bar 124 to the basal implants may be used without departing from the scope of the present invention.

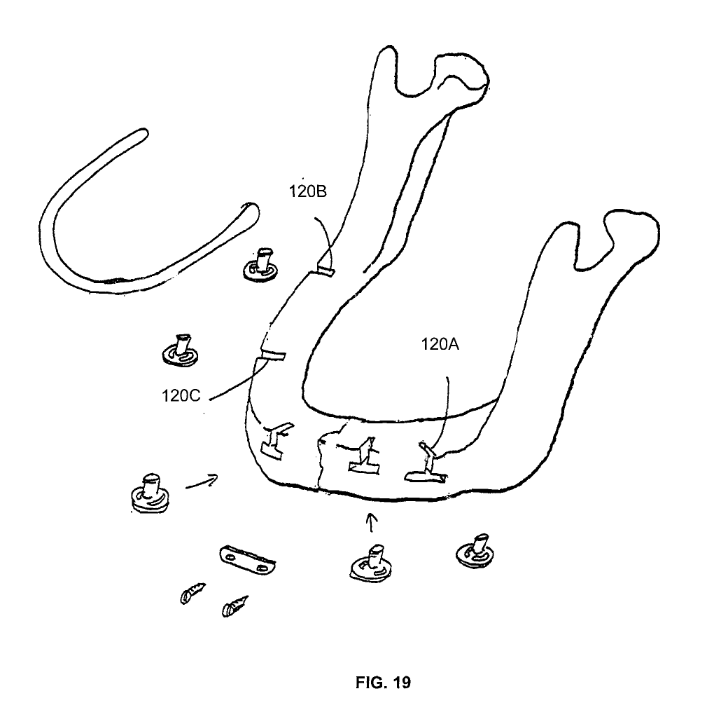

FIG. 19 illustrates the possibility of increasing the number of implants used, in the event the medical practitioner, in his judgment, sees that a better result can be obtained with more anchors.

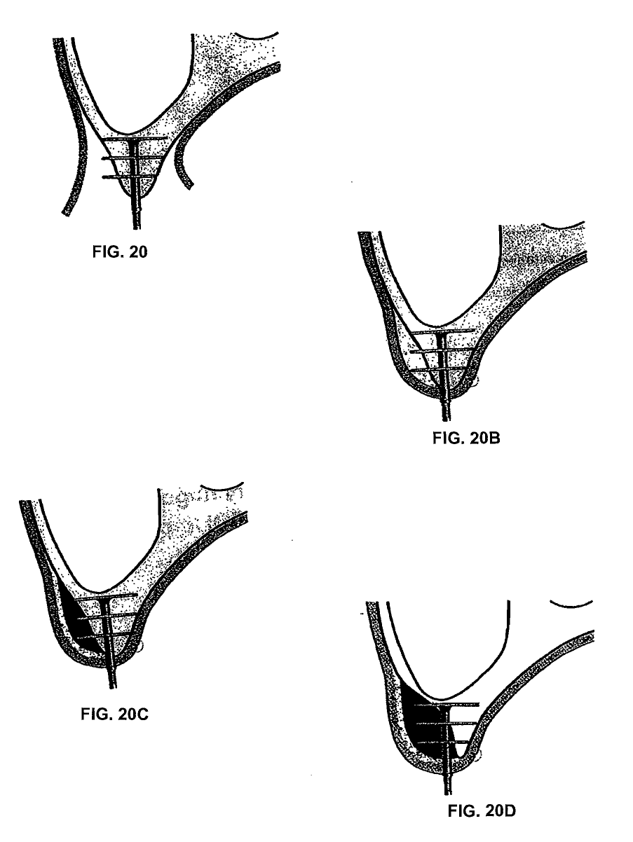

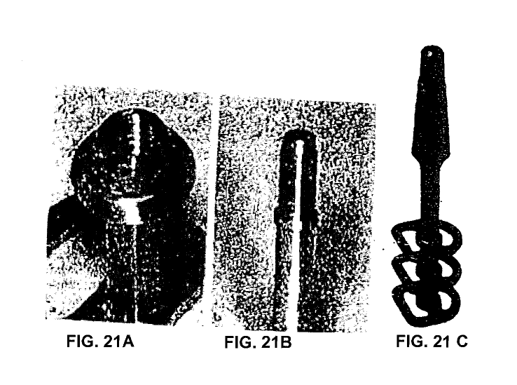

FIG. 20 show another aspect of the invention regarding alveolar augmentation. A considerable part of the vestibular alveolar wall of the jaw bone is sometimes removed before insertion of the lateral implant. This part of the bone is replaced by a resorbable or non-resorbable bone substitute. The substitute is granules of Hydroxylapatite or derivates thereof. The placement of the BOI implant is accompanied by the lateral augmentation in one surgical step. Disinfectant (e.g. iodine solution or derivates of iodine) may be mixed with the bone substitute in order to protect it from getting infected. Single stage implants penetrate into the oral cavity right after the operation. Two stage implants are covered by mucosa after operation and after healing phase, it is uncovered in a second surgical step before the teeth are mounted. If single stage implants are used, the diameter of the vertical implant part is considerably smaller than the diameter of the tooth-connecting platform; this way the entrance for bacterial invasion towards the bone augmentation area is small. Typical diameters may include a vertical part of 1.8-2.3 mm; Platforms for connection (for different types, see the book “Principles of BOI” by Stefan Ihde, incorporated by reference herein), may be 2.2-4.4 mm, with the vertical part being always thinner than the connection part, may it be an external thread (FIG. 21B) or an internal connection (FIG. 21A) or a one piece implant or (FIG. 21C)—that the augmentation material is a carrier for disinfectants and/or antibiotic medication.

The vestibular wall of the alveolar process is very prone to resorption. This is natural, but also may be exacerbated by surgery. So the danger is, that this part of the jaw bone will go away too soon. If we take it away right away and replace it by non-resorbable material, new bone will form in the area of the bone replacement material and since the material will not be subject to osteonal remodeling and resorption, it will stay a long time (longer than the natural alveolar wall would have stayed). Also, often infections stemming from teeth are caught inside the alveolar wall even after the teeth have been extracted. If the outer wall is removed before insertion of implant, all infection can flow out of the bone during or very soon after the operation. The infection can be controlled by the disinfectant or antibiotics which are held in place by the augmentation material. (Augmentation materials are very prone to infection, because they have no natural blood supply.)

It is also within the scope of the present invention for the lateral implant to be inserted only partly into native bone, for the lateral implant to serve as a “tent” for augmentation materials, for augmentation materials to be placed in the voids of the implant and/or for outside baseplates to hold away the periost and/or the membrane.

After insertion of a triple BOI implant, large portions of the crestal disks remain outside the bone. The defect can be closed with a mixture of a fibrin membrane and B-TCP. A paracrestal incision is recommended to ensure that the site is tightly closed. A possible alternative to filling with fibrin membranes is the placement of a rigid HA/polylactide membrane, followed by folding back the mucoperiosteal flap. The augmentation procedure may be performed also at any time later, after the implant is integrated. In the case shown here augmentation is an option. If, for reasons of space or because the residual ridge is too small, the threaded pin is not covered by native bone, placement of a membrane is necessary to prevent the soft tissues from growing into the space between the threaded pin and the original cortical bone. In some cases, especially in the distal mandible, the threaded pin may run parallel to the ridge outside the mucosa.

Reconstructive Surgery

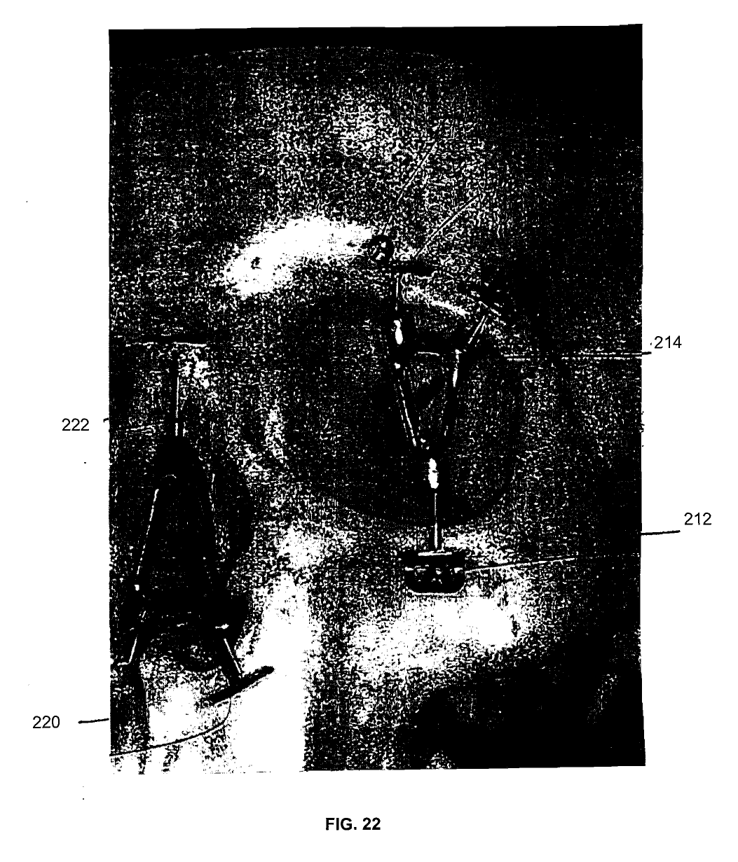

Referring now to FIG. 22, a perspective of a model human skull, includes a left eye socket A and a nasal cavity B. The eye socket A shows two upper lateral implants 210 as installed. Also shown is a lower implant 212 as installed. The implants each include a shaft which extends into the open space into which the anchor for a prosthetic device is to be maintained. In the eye socket A of FIG. 22, a mesiostructure or bridge 214 has been attached to and is maintained in position by the shafts of each of the three implants. Optionally, a short anchoring screw 216 may be used. Bending over some part of the struts created an improved primary stability for the implant, a lateral base of a lateral implant may be bent by an installing surgeon as depicted at 212, in order to achieve and maintain a desired positioning of a shaft of that implant. Also bent over parts of the implant are easily accessible for screw fixation.

Also visible in FIG. 22 are the T-shaped slots 220 that are cut by a surgeon with known surgical instruments before insertion of the lateral implants. These are shown in an oblique view proximate to the nasal sinus. Also visible in the nasal sinus are the shafts 222 of the implants used therein.

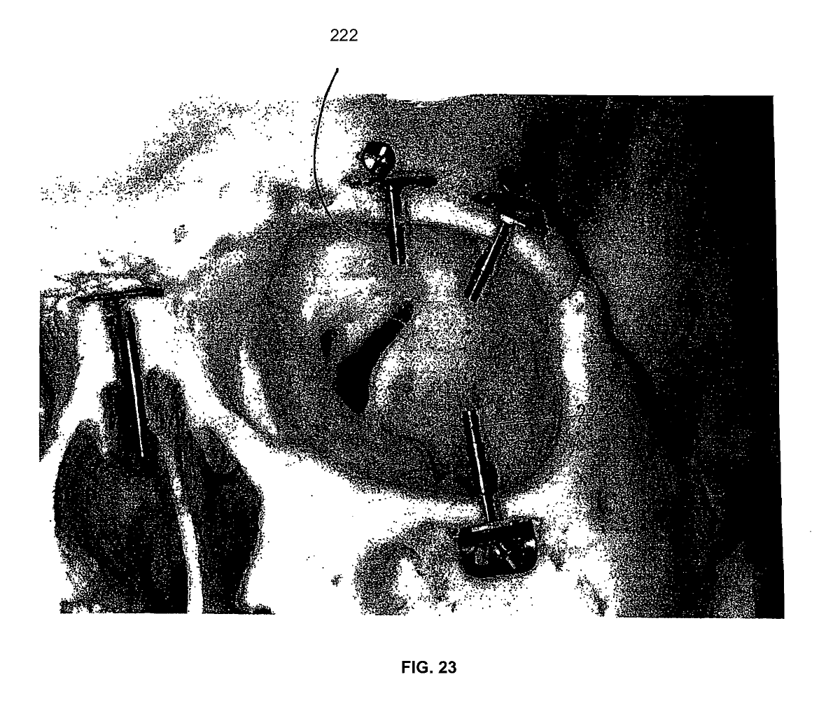

FIG. 23 also shows the shafts 222 of the implants as they appear after implant installation and before mounting of the bridge between them.

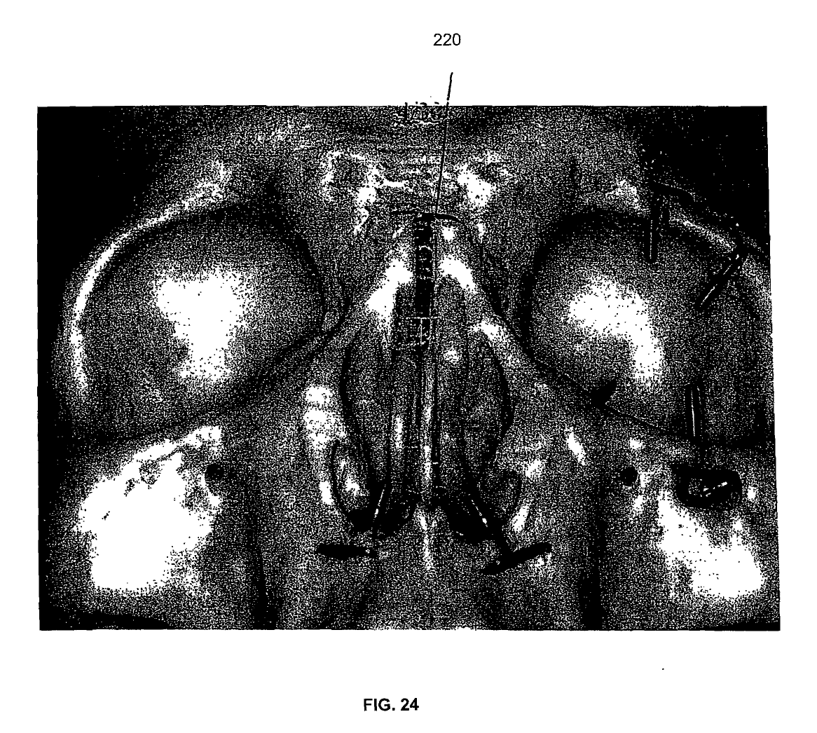

FIG. 24 is a front view. Again the lateral slots 220 are advantageously shown. In FIG. 24 the prosthetic anchor for the nasal sinus has had its mesiostructure or bridge installed 224.

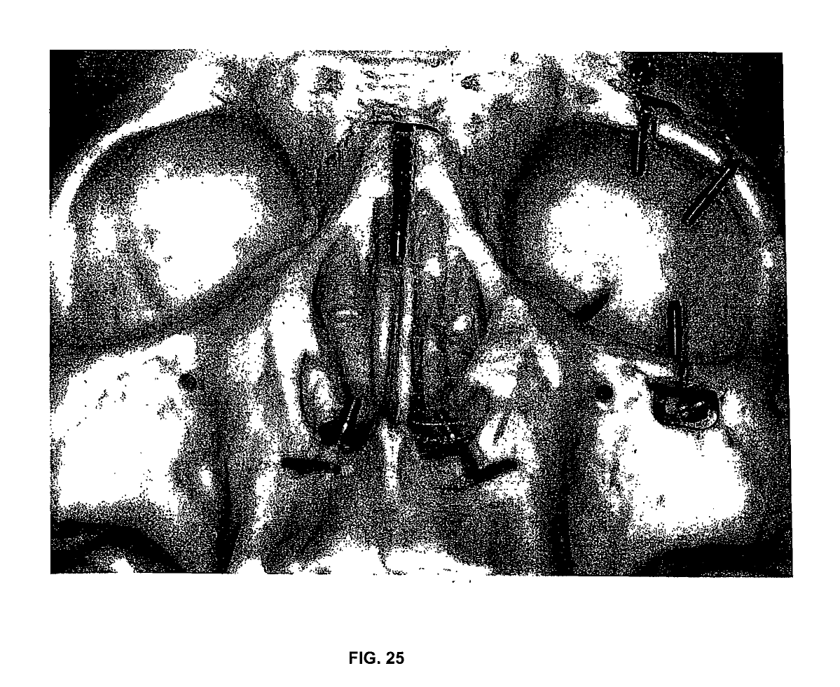

In FIG. 25 the nasal sinus is shown immediately after installation of the implants and before installation of the bridge.

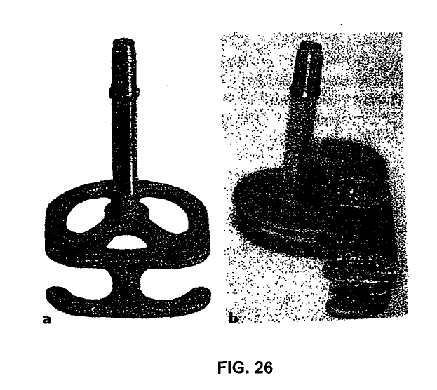

FIG. 26 shows a variety of lateral implant configurations that may be used. These lateral implants are each comprised of at least a base section and a shaft.

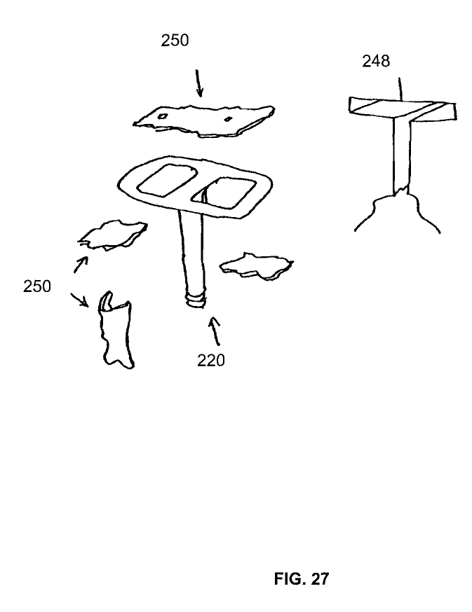

FIG. 27 shows a lateral implant, T-shaped slot and fibrin membrane in an exploded view to show their relative positions. Clearly, after a T-shaped slot 248 has been created by the surgeon, the lateral implant will be inserted into it. In order to promote healing, a fibrin membrane 250 may be used. This fibrin membrane may be installed in any one of the positions shown, at the surgeon's discretion. The fibrin membrane or cloth increases the quantity of woven bone available for early healing and it helps sealing the operation site for a good wound closure. Also the fibrin membrane or the fibrin cloth traps a large number of Thrombozytes, which promote osseous healing. This way the need of (difficult) preparation and application of Thrombocyte-concentrates are eliminated.

In operation, during or shortly after a facial resection to remove a tumor or other surgery, the prosthetic device anchor is installed as follows: a T-shaped saw is used to create a T-shaped slot in the patient's bone immediately proximate to the area into which the prosthesis is to be installed. Thereafter, a lateral implant is installed in the slot. Optionally, a fibrin membrane may be installed in the slot between the lateral implant and the bone to promote healing and woven (callus) bone development. Any number of implants may be used, but in the depicted embodiment three implants are used. A mesiostructure comprising a bridge, bar or the like is attached to the shaft of the implant(s). Attachment may be by any mechanical means including slipping on axially, screwing on or bending wings around the mesiostructure around the shaft of the lateral implant.

In the event the lateral implants may, in the surgeon's discretion, require additional anchoring, a base element of the implant may be bent over where the base extends from the T-shaped slot. A further option is to add a short screw over the slot to hold the lateral implant in place.

The system, apparatus and method of the present invention is particularly well-suited for creating anchor points for orbita, epitheses or prosthesis, insertion of the implants into the supra orbital margin of the os frontalis and the infra orbital margin of the os zygomaticun, insertion of the implants into the anterior floor of the nose, into the maxillary bone, into the squama frontalis of the os frontale or the upper maxilla. In the depicted embodiment, two of the implants are used in the lower bone margin and one in the upper margin of the orbita.

Additional bone screws to secure the base plates in the bone and against extractive forces before a final integration are covered with a skin flap or skin graft and are thereby protected from infection due to their isolation from the environment.

Clip-type lateral implants will double or triple vestibular anchorage, as depicted in FIG. 26, are advantageously used where space is limited, and may aid in avoiding breaking into the cranium or the sinuses.

The use of the autologous fibrin cloths or membranes for covering the penetration area of the vertical implant part underneath the skin before reflection and suturing enhances healing and creates protection with blood coagulum and also helps to avoid infection.

Optionally, the implants and assembly may be installed at any time from the resection itself through and during radiation treatment or shortly thereafter. In the depicted embodiment, the base of the lateral implants are 7-12 millimeters in either dimension.

Further advantages over the prior art are that lateral implants distribute the forces to bone areas which are strong (highly mineralized), as opposed to prior art pins that come out of the bone on areas that are not so strong (low mineralization areas) and where screws would not adequately hold. Of course, the present system may be used in combination with conventional screws for fixation. The open slots of the present system promote woven bone formation, especially in osteoporotic bone. Woven bone is created in addition to the existing cortical bone, so there is a more bone in the end. The pins used herein may be completely smooth so infection can not catch easily as in screw implants.

Coatings

Stabilization of the implants herein may follow a fundamentally different approach than the previously known techniques that, without exception, use substances that accelerate and stimulate formation of new bone tissue to stabilize enossal implants.

Substances are known that hinder or prevent the internal formation of new bone, which is known as remodeling. Such substances are used, for instance, to treat osteoporosis if there is a need to delay bone deterioration caused by remodeling. These substances affect the activity of the so-called osteoclasts. They reduce the activity, propagation, or motility of the osteoclasts, the cells that degrade bone. At the state of the art, those substances are administered orally or parenterally for general medical problems (such as osteoporosis).

These substances have substantial adverse effects in the area of implantology, though, if they are administered in that manner. For instance, very severe inflammations can occur after implantation, as in patients who have received enossal dental implants or surgical-orthopedic implants. The most feared complications are the notorious osteomyelitis (inflammation of the bone marrow) and osteonecrosis (death of bones without bacterial action). For those reasons, implantations in patients who are taking such substances for general medical reasons are now considered highly risky and essentially contraindicated. The reason is quite simple: Because of the reduced activity of the osteoclasts, the bone is less ossified. As a result, it is more strongly mineralized and the blood supply that is important for defense is lacking. Now if such a damaged bone is exposed to surgery, unintended penetration of bacteria into the bony surgical field can occur. Then, because the blood supply is inadequate, those bacteria cannot be repelled by the body's immune system, and they can propagate.

Even entire regions of bone can die in the same manner under therapy with substances that prevent or hinder osteonal remodeling. It is often not realized that the bone is dead, because dead bone retains its structural integrity for a long time, and even when dead, can transfer force and appear as a morphologic structure. One to two million load cycles with a load appropriate for the bone are required before a dead bone yields structurally from the effects of an alternating load. If one considers that the leg of an adult carries out only about 5,000 steps, that is, only 5,000 load cycles a day, the great potential lasting strength of even dead bone tissue becomes clear.

On the other hand, overloaded bone with microdefects due to the use of substances that inhibit or prevent osteonal remodeling, plus the repair damages that are harmful with respect to structural integrity, breaks after only about six weeks.

Surprisingly, though, the controlled histological studies on which the invention is based show that the severe side-effects of the substances that prevent the osteonal system from developing and functioning can be avoided for the region of the implant by not administering those substances orally or parenterally, but locally as part of the actual surgery, microtherapeutically in a sense. Coating of the implant with the active substance according to the invention is an advantageous form of application. As many implant surfaces have a certain roughness in any case, application of such a coating is not a problem.

It has also been found that both fat-soluble and water-soluble substances can be used equally well. Thus, a great range of substances can be used for the solution according to the invention: beyond the biphosphonates—namely etidronate, clodronate, tiludronate, pamidronate, alendronate, risedronate, ibandronate, and zoledronate—estrogens, TGF-beta, gallium nitrate, Plicamycin, Calcitriol, Calcetonin, and Bafilomycin are also materials suitable for implant coating according to the invention.

As a result of the coating according to the invention, there is a situation near the enossal implant surface in which the bones exhibit no spatially limited repair signs. Thus, the implants also remain stable. The concentration of the substances used for the coating according to the invention decreases with time. They are diluted by the liquid circulating in the bones and by the blood flow, so that their concentration decreases below the threshold of therapeutic activity and regular remodeling slowly becomes possible again. By that time, though, the implants are finally well integrated into the bone and damages from use (microcracks) which act on the bones can no longer accumulate with time with repair defects. The repair also proceeds more slowly.

It can be advantageous to combine the substances named above with antibiotics to fight any local infections that might occur, or to prevent them prophylactically.

One particularly advantageous combination of the coating provided is that of a biphosphonate (such as Ibandronate) with an antibiotic (such as tetracycline). Bafilomycin alone, on the other hand, can develop both effects. In appropriate concentration, it acts as an antibiotic and also as an inhibitor of osteonal remodeling.

The enossal surface of a dental or surgical (screw) implant may be given a microporous surface structure by known processes, such as sandblasting, etching, or a combination of both of those processes, or by sintering titanium beads onto it. Then an adhesive water-soluble or fat-soluble solution of Ibandronate is applied, by which the active substance is distributed over the enossal surface of the implant in a total amount of 3 to 40 mg.

In searching for substances that can be used in the implant region to reduce osteonal activity in the vicinity of implants, but which are not toxic, we quite surprisingly found the following: even a thin coating with ordinary sodium chloride has such a local inhibitory action on the osteoclastic activity involved in remodeling. Such a coating can be produced by immersing the implant (with a roughened surface, if possible) in a sodium chloride solution (such as physiological, 0.9%, sodium chloride) at the end of the cleaning procedure and then drying it carefully. Then a thin coating of pure sodium chloride remains on the surface. This layer dissolves in the fluid and in the local blood during and after setting of the implant. That produces a site of higher salt concentration in the bone, which limits the implant. Histological examinations show that this concentration influences the remodeling. It is not sufficient for just the usual physiological solution of sodium chloride to be present. The concentrations in the surrounding bone must be far higher than those that occur physiologically in the blood. The same is true for a thin, soluble coating with CaP, CaSO₄, and other bone substrate substances which exhibit an action similar to that of sodium chloride. It is the massive local elevation of the concentration of these substances and the rapid solubility of the substances that is critical. Thus they cannot just be present on the surface (as, for instance, the older CaP coating intended to be permanent, or earlier hydroxyl apatite coatings).

A high ion concentration is generated around the implant by means of the substances mentioned above, preventing remodeling for a certain period: until the implants become orthopedically splinted by the prosthesis. If one selects non-toxic, degradable substances, they can easily be degraded later, so that the long-term osteopetrotic effect ceases and the pen-implant bone regenerates normally with time.

A typical example would be a thin crust from pure Sodiumchloride including a Biphosphonate, which is manufactured by dissolving the biphosphone in Sodiumchloride sulution, applying it to the implant surface and then drying the surface carefully. This way an even distribution of almost pure, medication-loaded Sodiumchloride is created. After insertion of the implant, the high concentration of Sodiumchloride will dissolve and the high gradient of concentration will be lowered by fluctuation through the Haversian canals. Together with the Sodiumchloride the drug will be transported along passively, although its concentration would never be enough to cause this dissolution or fluctuation.

Structure and Materials

In order that the vertical cut in the bone for receiving the shaft of the implant be kept as narrow as possible for rapid healing, the shaft of the implant according to the invention has an oval to elliptical profile cross-section. Its diameter D, which simultaneously forms the longitudinal axis of the shaft profile and which is arranged in the direction of insertion of the implant, is greater than 2.0 mm, and its diameter d, measured across the smaller axis of the profile, is less than 2.0 mm. In one preferred embodiment, the diameter D is 2.3 mm while a diameter of 1.9 mm is selected for diameter d. In a long bone the dimensions would be larger; for example a small axis less than 4.0 mm and a large axis greater than 4.0 mm.

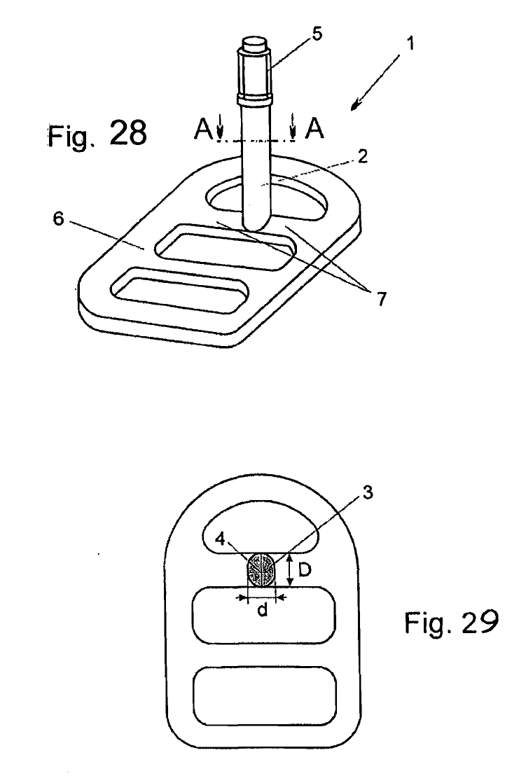

FIGS. 28 and 29 are perspective and top views showing the non-circular post.

Because of the shape of the shaft profile according to the invention, the vertical opening that must be made surgically in the jaw bone can be chosen relatively small. Therefore relatively narrow vertical slots are ground in the jawbone for insertion of the implant. They close rapidly through the natural healing process. That is particularly advantageous for implants in the upper jaw.

Use of bone replacement material, which formerly had to be used to close wide vertical openings, is minimized because the newly forming bone tissue bridges over openings less than 2.0 mm in the jaw bone in a very short time, often closing directly or by way of network bone.

On the other hand, the cross-section of the shaft that bears and transfers the load is not reduced, because of the oval to elliptical cross-section of the profile. In spite of the smaller diameter d of the profile cross-section, which must be selected relatively narrow so that the implant can be inserted through a relatively narrow slot in the jaw bone, the danger of breakage of the shaft with the profile cross-section according to the invention is not increased. For instance, the number of load cycles to breakage (for a diagonal load) in fatigue tests is doubled with the implant shaft according to the invention.

A further advantage of the profile cross-section according to the invention is seen in the fact that the forces caused by chewing are transferred more evenly to the implant base and into the jaw bone by the oval to elliptical profile of the shaft.

The oval to elliptical cross-section according to the invention can extend over the entire free length of the shaft to below the threaded end of the shaft, passing then into a circular cross-section; or it can be provided only in the partial segments of the implant shaft which are in the jaw bone after insertion of the implant.

The oval shaft offers a further advantage for basal implant with round base disks. These base disks are not secure against rotation, and can easily turn in the bone. The oval shape of the vertical part of the implant provides security against rotation for those implants, also. That is highly advantageous in clinical use.

The invention is explained briefly in the following by means of an example embodiment. The accompanying drawing shows:

FIG. 28. A schematic representation of the implant according to the invention. FIG. 29 depicts the section A-A as indicated in FIG. 28.

According to the subject of the present invention, the implant 301 comprises the implant foot 306, which can, for instance, be designed as a disk or a ring, and a shaft 302, connected to the implant foot by pins 307. Shaft 302 itself can be made as a simple cementing post, or provided with a threaded end to hold and fasten the structural part of a dental prosthesis.

Shaft 302 of the implant 301 has, according to the invention, an oval to elliptical profile cross-section 303. It is arranged in relation to the implant food 306 so that the longitudinal axis 304 of the profile cross-section 303, or the outside diameter D, lies in the direction in which implant 301 is forced into the previously prepared implant bed on insertion of the implant. The profile cross-section 303 according to the invention of the shaft 302 can extend over the entire free length of shaft 302 into the vicinity of the end of the shaft which, in the present example, is provided with a thread 305, or it can be provided only in the section of shaft 302 adjacent to the implant foot 306, which is in the jaw bone after insertion of the implant 301.

According to a preferred embodiment, the outside diameter D of the shaft profile is 2.3 mm, while the diameter d is 1.9 mm.

So as to grind out the narrowest possible vertical slot for holding and passage of the implant in the jaw bone, the diameter d of profile cross-section 303 should be less than 2.0 mm, and diameter D greater than 2.0 mm, depending on the chewing forces that must be transferred to the jaw bone.

It is known that certain textures may be used for various metal implants in order to promote osseointegration. However, such surfaces having pores or microstructural texture are not always optimally sanitary. For fighting infection where metal implants touch tissue, gum or skin, a more perfectly smooth surface, for example stainless steel is less likely to harbor bacteria and cause infection. Accordingly, it is another aspect of the present system, apparatus and method that the base disk of the implant be made of a first material or have a first texture in that at least the outwardly extending portion of the shaft or post of the same implant be made of a second material or have a second texture. One or both of the basal implant parts may consist of titanium or its alloys, which may be advantageously used for osseointegration on the base disk part of the implant. The vertical implant part may advantageously be made of steel, CoCrMo compound, CoCr in an alloy with other bio compatible materials, zirconium or a zirconium compound. The structure may also advantageously fuse the basal part of the implant and the vertical part with laser welding, by riveting, by locking a retaining cone by mechanical pressing, by screwing together, with or without a lock, by pins. It may advantageously be structured that the connection between the basal and vertical parts of the implant may be reversible. It may also be advantageously structured such that the basal and vertical parts of the implant may be made of a uniform core material with a surface coating for the basal part that is different than that for the vertical part, more particularly that the surface of the basal part is textured for osseointegration while the outwardly extending portion of the vertical part is smooth.

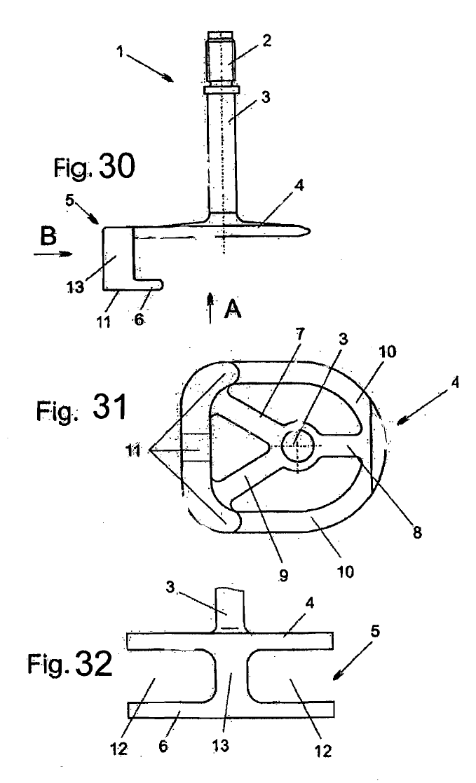

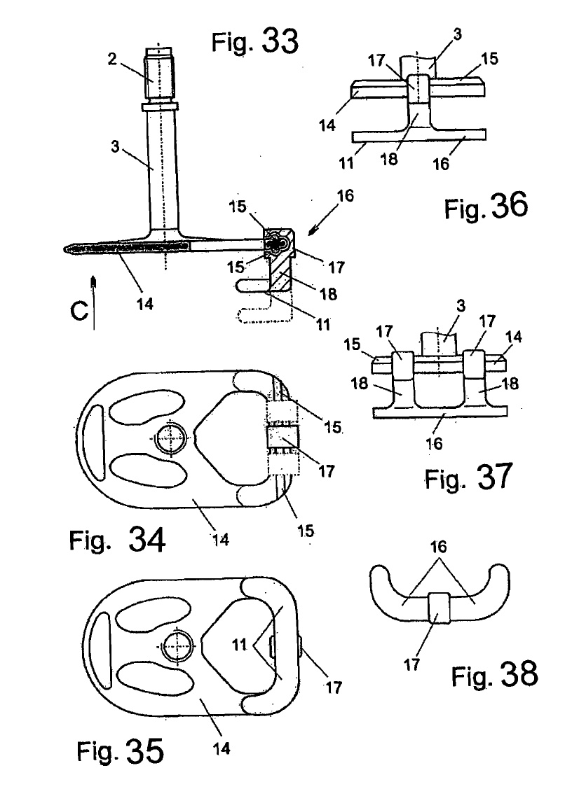

FIGS. 30-38 depict a base plate with an additional partial plate, which may be optionally added by the doctor to allow him greater flexibility in fitting an individual's anatomy.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

1.-37. (canceled)

38. A reconstructive stabilizer capable of prosthesis anchoring comprising:
at least two basal implants, each of said implants comprising a shaft and a base, said base being substantially orthogonal to said shaft;
said shaft having an in-bone length and an out-of-bone length, said in-bone length being substantially equal to or less than said out-of-bone length;
said at least two basal implants being configured to comprise anchor points upon installation;
a mesiostructure mountable on said anchor points to span a cavity in a bone, such that the bone defining the cavity is stabilized.

39. The stabilization implant of claim 38 further comprising;
a third basal implant wherein said mesiostructure is mountable on a shaft of said third implant, as well as on said shafts of said first two implants.

40. The stabilization implant of claim 38 wherein said mesiostructure is attached to said shafts of said basal implants by at least one of a slide, a screw, or a binding wing.

41. The stabilization implant of claim 38 further comprising a base anchoring element comprising at least one of a screw or a bendable extension of said base.

42. The stabilization implant of claim 38 further comprising said bases incorporating clip type anchors.

43. The stabilization implant of claim 38 further comprising said basal implants being dimensioned to be installed in a T-shaped slot in a highly mineralized bone area.

44. The stabilization implant of claim 38 wherein said mesiostructure is configured to support a prosthesis.

45. The stabilization implant of claim 38 wherein the cavity being stabilized is one of an eye socket or a sinus.

46. The stabilization implant of claim 38 wherein said mesiostructure is generally triangular.

47. A stabilization implant comprising:
at least three basal implants, each of said implants comprising a shaft and a base, said base being substantially orthogonal to said shaft;
said basal implants being configured to implant in T-shaped slots in bones surrounding a cavity defined by the bone; and
a mesiostructure mountable on said shafts of said at least three basal implants such that the bone defining the cavity is stabilized.

48. The stabilization implant of claim 47 wherein said mesiostructure is attached to said shafts of said basal implants by at least one of a slide, a screw, or a binding wing.

49. The stabilization implant of claim 47 further comprising a base anchoring element comprising at least one of a screw or a bendable extension of said base.

50. The stabilization implant of claim 47 further comprising said bases incorporating clip type anchors.

51. The stabilization implant of claim 47 further comprising said basal implants being dimensioned to be installed in a T-shaped slot in a highly mineralized bone area.

52. The stabilization implant of claim 47 wherein said mesiostructure is configured to support a prosthesis.

53. The stabilization implant of claim 47 wherein the cavity being stabilized is one of an eye socket or a sinus.

54. The stabilization implant of claim 47 wherein said mesiostructure is generally triangular.
